(19) United States
(12) Patent Application Publication
(10) Pub. No.: US 2009/0215841 A1
(43) Pub. Date: Aug. 27, 2009

(54) D2 Receptor Ligand-078

(75) Inventors: William C. Blackwell, III, Cary, NC (US); James Hulsizer, Wilmington, DE (US); JianWei Liu, Wilmington, DE (US); Gary Steelman, Wilmington, DE (US); Rebecca Urbanek, Wilmington, DE (US); Dan Widzowski, Wilmington, DE (US); Ye Wu, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/388,548

(22) Filed: Feb. 19, 2009

Related U.S. Application Data
(60) Provisional application No. 61/030,332, filed on Feb. 21, 2008.

(51) Int. Cl. A61K 31/428; C07D 277/60; A61P 25/00

(52) U.S. Cl. 514/366; 548/150

(57) ABSTRACT

This invention relates to the compound of Formula I and pharmaceutically acceptable salts thereof:

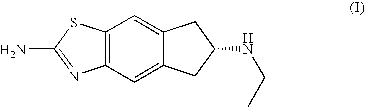

(R)-N*6*-ethyl-6,7-dihydro-5H-indeno[5,6-d]thiazole-2,6-diamine
This invention also relates to methods of making, methods of using, and pharmaceutical compositions comprising the compound of Formula I and salts thereof.

### CROSS-REFERENCE TO RELATED PATENT APPLICATION

This patent claims priority to U.S. Provisional Patent Application No. 61/030,332 (filed Feb. 21, 2008). The entire text of that patent application is incorporated by reference into this patent.

### FIELD OF THE INVENTION

This invention relates to a novel compound and its use as an antipsychotic. In particular, the invention relates to a compound (and salts thereof) having dopamine D2 receptor partial agonistic activity, methods of preparing the compound and its salts, and uses of the compound and its salts for therapeutic and drug screening purposes.

### BACKGROUND OF THE INVENTION

Clinicians regularly use antipsychotics that block dopamine D2 receptors. Antipsychotics are often classified as “typical” and “atypical” antipsychotics. Atypical antipsychotics generally have a lower incidence of side effects compared to typical antipsychotics. Only a few dopamine-depleting agents, other than those that provide D2 receptor blockade, achieve antipsychotic activity. Such agents include, for example, reserpine and α-methyl-para-tyrosine. Moderate to severe side effects (e.g., poor tolerability) remain problematic with clinically prescribed antipsychotics. For example, extrapyramidal side effects (“EPS”) and/or elevation of prolactin limit the number of patients who can take some current medications and decrease patient compliance. For some D2 antagonist drugs (e.g., amisulpride and risperidone), hyperprolactinemia can lead to secondary problems, such as galactorrhoea, gynaecomastia, breast pain, and amenorroea.

There is, therefore, a need for new effective antipsychotic drugs with reduced side effects and improved tolerability.

### SUMMARY OF THE INVENTION

Briefly, this invention is directed, in part, to the compound of Formula I and salts thereof:

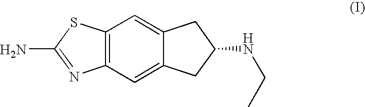

### (R)-N*6*-ethyl-6,7-dihydro-5H-indeno[5,6-d]thiazole-2,6-diamine

The compound of Formula I has been identified as a ligand of the dopamine D2 receptor, with a binding Ki of approximately 150 nM for the dopamine D2 receptor. It has been observed to have antipsychotic activities in animal D-amphetamine induced locomotor activity and conditioned avoidance response assays. It is believed that the compound of Formula I and salts thereof (particularly pharmaceutically acceptable addition salts) have valuable pharmacological properties, particularly with respect to the effect on the dopaminergic system of the central nervous system.

This invention also relates, in part, to pharmaceutical compositions comprising the compound of Formula I or a pharmaceutically acceptable salt thereof and, optionally, one or more pharmaceutically acceptable carriers and/or diluents.

This invention also relates, in part, to the use of the compound of Formula I or a pharmaceutically acceptable salt thereof to prepare a pharmaceutical composition comprising the compound of Formula I or salt thereof and, optionally, one or more pharmaceutically acceptable carriers and/or diluents.

This invention also relates, in part, to the use of a compound of Formula I or a pharmaceutically acceptable acid salt thereof for preparing a drug having an effect on the dopaminergic system of the central nervous system, such as for treating dopamine-receptor-related central nervous neuro-psychiatric conditions.

This invention also relates, in part, to a process for making a pharmaceutical composition characterized in that a compound of Formula I or a pharmaceutically acceptable salt thereof is incorporated with one or more inert carriers and/or diluents by a non-chemical method.

Further benefits of Applicants' invention will be apparent to one skilled in the art from reading this specification.

### BRIEF DESCRIPTION OF THE DRAWINGS

FIG. 1 is a graph depicting the agonist effect of compounds on D2-CHO cells using CDS.

FIG. 2a is a graph depicting the effect of a compound of Formula I on D-amphetamine hyperlocomotion in habituated rats.

FIG. 2b is a graph depicting the effect of comparative compound C3 on D-amphetamine hyperlocomotion in habituated rats.

FIG. 3a is a graph depicting the results of a Conditioned Avoidance Responding (CAR) Assay employing aripiprazole.

FIG. 3b is a graph depicting the results of a CAR Assay employing a compound of Formula I.

FIG. 3c is a graph depicting the results of a CAR Assay employing haloperidol.

FIG. 3d is a graph depicting the results of a CAR Assay employing comparative compound C3.

FIG. 4a is a graph depicting the results of a Catalepsy Mice Model employing a compound of Formula I.

FIG. 4b is a graph depicting the results of a Catalepsy Mice Model employing comparative compound C3.

FIG. 5 shows the molecular structure of compound of Formula I as the R isomer.

### DETAILED DESCRIPTION OF PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

This invention is directed to the compound of Formula I and salts thereof (particularly pharmaceutically acceptable salts):

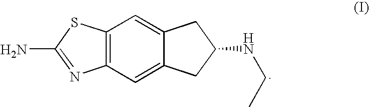

The compound of Formula I has been observed to have properties not shared by other structurally similar compounds, such as the (S)-enantiomer and compounds wherein the ethylamine moiety of Formula I is replaced with other mono- or di-alkyl amino moieties.

During the course of research on D2 ligands, the compound of Formula I was identified and discovered to possess unexpected D2-mediated properties over its enantiomer and other analogs. The compound of Formula I has been observed to possess relatively potent antagonism of D2 receptors in combination with a measurable level of D2 partial agonism. The D2 partial agonism is believed to mitigate D2-mediated side effects, such as hyperprolactinemia and EPS. An exemplary D2 partial agonist is aripiprazole, marketed under the name Abilify®. Aripiprazole has shown less propensity to cause hyperprolactinemia than antipsychotics (e.g., risperidone and haloperidol) having D2 antagonist properties without partial agonism.

The compound of Formula I (or a pharmaceutically acceptable salt thereof) generally may be used in a method for treating mammals, especially humans, suffering from dopamine related central nervous system disorders (e.g., schizophrenia; Parkinson's disease; Tourette's Syndrome; hyperprolactinemia; and drug abuse, such as abuse of alcohol or cocaine) by administering a therapeutically effective amount of a compound of Formula I or a salt thereof. Other contemplated central nervous system disorders that may be treated include, for example, major depressive disorder (“MDD”) and bipolar disorder.

Pharmaceutically acceptable salts include salts that are useful for administering the compound of Formula I to a patient. Pharmaceutically acceptable salts also include useful salts that the compound of Formula I may form in vitro or in vivo. Pharmaceutically acceptable salts include various acid addition salts, such as, for example, hydrochloric, hydrobromic, sulfuric, phosphoric, lactic, citric, tartaric, succinic, maleic, and fumaric acid salts. Alkyl sulfonic acids (e.g., CH₃SO₃ H) also are generally suitable for making pharmaceutically acceptable salts. In general, a pharmaceutically acceptable salt has one or more benefits that outweigh any deleterious effect that the salt may have.

A pharmaceutical composition may be prepared by admixing a compound of Formula I or a pharmaceutically acceptable salt thereof with a pharmaceutically acceptable carrier to achieve a pharmaceutical preparation comprising a therapeutically effective amount of the compound of Formula I per unit dose.

Compositions comprising a compound of Formula I or a pharmaceutically acceptable salt thereof can be prepared for administration to humans and other vertebrates in unit dosage forms, such as, for example, tablets, capsules, pills, powders, granules, sterile parenteral solutions or suspensions, oral solutions or suspensions, oil-in-water and water-in-oil emulsions, and suppositories. For oral administration, either solid or fluid unit dosage forms can be prepared. For preparing solid compositions (e.g., tablets), the compound or a pharmaceutically acceptable salt thereof can be mixed with conventional ingredients such as, for example, talc, magnesium stearate, dicalcium phosphate, magnesium aluminum silicate, calcium sulfate, starch, lactose, acacia, methylcellulose, and functionally similar materials that act as pharmaceutical diluents or carriers. Capsules may be prepared by mixing the compound or a pharmaceutically acceptable salt thereof with an inert pharmaceutical diluent, and filling the mixture into a hard gelatin capsule of appropriate size. Soft gelatin capsules may be prepared by machine encapsulation of a slurry of the compound (or a pharmaceutically acceptable salt thereof) with an acceptable vegetable oil, light liquid petrolatum, or other inert oil.

Fluid unit dosage forms for oral administration, such as syrups, elixirs, and suspensions, can be prepared by, for example, dissolving the compound or salt in an aqueous vehicle together with sugar, aromatic flavoring agents, and preservatives to form a syrup. Suspensions can be prepared with an aqueous vehicle with the aid of a suspending agent such as acacia, tragacanth, methylcellulose, and the like.

For parenteral administration, fluid unit dosage forms can be prepared utilizing the compound or a pharmaceutically acceptable salt thereof and a sterile vehicle. In preparing solutions, the compound or a pharmaceutically acceptable salt thereof can be dissolved in water for injection and filter-sterilized before filling into a suitable vial or ampoule, and sealing. Adjuvants, such as a local anesthetic, preservative, or buffering agent, can be dissolved in the vehicle as well. The composition can be frozen after filling into a vial and the water removed under vacuum. The resulting lyophilized powder can then be scaled in the vial and reconstituted before use.

The compound and pharmaceutically acceptable acid salts thereof generally have valuable pharmacological properties, particularly an effect on the central nervous system, including a stimulating effect on the dopamine receptors (either of, or both of, the autoreceptors and the postsynaptic receptors) or an inhibiting effect of the dopamine receptors, thus providing partial agonistic activity. The compound and salts thereof having a high intrinsic efficacy for the dopamine receptors in the CNS of mammals are contemplated to be suitable for treating Parkinson's disease, either in mono-therapy or in combination therapy with, for example, L-DOPA and carbidopa. The compound and salts also are contemplated to be anti-hyperprolactinergic drugs. The compound and salts having a low intrinsic efficacy (partial agonists, inverse agonists, and/or antagonists) for the dopamine receptors in the CNS of mammals are contemplated to be suitable for treating psychotic disorders, such as schizophrenia.

The compound of Formula I or a pharmaceutically acceptable salt thereof can be administered to treat conditions mentioned herein. The exact dosage and frequency of administration will depend on the particular condition being treated; the severity of the condition being treated; the age, weight, and general physical condition of the particular patient; other medication the patient may be taking; and various other factors known to those skilled in the art. Thus, the compound or a pharmaceutically acceptable salt thereof, along with a pharmaceutically acceptable carrier, diluent, or buffer, can be administered in a therapeutic or pharmacological amount effective to alleviate the central nervous system disorder with respect to the physiological condition diagnosed. The compound or a pharmaceutically acceptable salt thereof can, for example, be administered intravenously, intramuscularly, topically, transdermally (e.g., by skin patches), buccally, or orally to man or other vertebrates as will be apparent to those of skill in the art.

The compound and pharmaceutically acceptable salts described herein are contemplated to be useful for treating neuropsychiatric disorders, including, for example, conditions associated with or leading to psychosis, emotional and behavioral disturbances, schizophrenia and schizophrenia spectrum disorders, psychotic disorders in the context of affective disorders, depression, psychosis disorders induced by drugs/medication (such as Parkinson's psychosis), drug induced movement disorders (dyskinesias in Parkinson's disease), psychosis and behavioral disorders in the context of dementias and psychotic disorders due to a general medical conditions, or combinations thereof.

The compound and pharmaceutically acceptable salts thereof are also contemplated to be useful for treating anxiety disorder(s), including, for example, panic disorder(s) without agoraphobia, panic disorder(s) with agoraphobia, agoraphobia without history of panic disorder(s), specific phobia, social phobia, obsessive-compulsive disorder(s), stress related disorder(s), posttraumatic stress disorder(s), acute stress disorder(s), generalized anxiety disorder(s), and generalized anxiety disorder(s) due to a general medical condition.

The compound and pharmaceutically acceptable salts thereof are also contemplated to be useful for treating mood disorder(s) including but not limited to a) depressive disorder(s), including but not limited to major depressive disorder(s) and dysthymic disorder(s); b) bipolar depression and/or bipolar mania including but not limited to bipolar i, including but not limited to those with manic, depressive or mixed episodes, and bipolar ii; c) cyclothymiac's disorder(s); and d) mood disorder(s) due to a general medical condition.

Treatment is contemplated to be achieved by administering to a patient or subject (e.g., a human or an animal such as a dog) in need thereof a therapeutically effective amount of a compound of Formula I or a pharmaceutically acceptable salt thereof. Pharmaceutical compositions comprising a compound of Formula I or a pharmaceutically acceptable salt thereof, together with one or more pharmaceutically acceptable carriers and/or diluents, can generally be used for therapeutic purposes.

The compound of Formula I or a pharmaceutically acceptable salt thereof or a pharmaceutical composition comprising a compound of Formula I or a pharmaceutically acceptable salt thereof may be administered concurrently, simultaneously, sequentially, or separately with another pharmaceutically active compound or compounds selected from the following:

(i) antidepressants including, for example, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robalzotan, sertraline, sibutramine, thionisoxetine, tranylcypromaine, trazodone, trimipramine, venlafaxine, and equivalents and pharmaceutically active isomer(s) and metabolite(s) thereof;

(ii) atypical antipsychotics including, for example, quetiapine and pharmaceutically active isomer(s) and metabolite(s) thereof;

(iii) antipsychotics including, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone, and equivalents and pharmaceutically active isomer(s) and metabolite(s) thereof;

(iv) anxiolytics including, for example, alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants including, for example, carbamazepine, valproate, lamotrogine, gabapentin and equivalents and pharmaceutically active isomer(s) and metabolite(s) thereof;

(vi) Alzheimer's therapies including, for example, donepezil, memantine, tacrine and equivalents and pharmaceutically active isomer(s) and metabolite(s) thereof;

(vii) Parkinson's therapies including, for example, deprenyl, L-dopa, Requip, Mirapex, MAOB inhibitors such as selegine and rasagiline, COMT inhibitors such as Tasmar(tolcapone), A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies including, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies including, for example, abciximab, activase, citicoline, crobenetine, desmoteplase, repinotan, traxoprodil and equivalents and pharmaceutically active isomer(s) and metabolite(s) thereof;

(x) urinary incontinence therapies including, for example, darafenacin, falvoxate, oxybutynin, propiverine, robalzotan, solifenacin, tolterodine, and equivalents and pharmaceutically active isomer(s) and metabolite(s) thereof;

(xi) neuropathic pain therapies including, for example, gabapentin, lidoderm, pregablin and equivalents and pharmaceutically active isomer(s) and metabolite(s) thereof;

(xii) nociceptive pain therapies such as celecoxib, etoricoxib, lumiracoxib, rofecoxib, valdecoxib, diclofenac, loxoprofen, naproxen, paracetamol, and equivalents and pharmaceutically active isomer(s) and metabolite(s) thereof;

(xiii) insomnia therapies including, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, roletamide, triclofos, secobarbital, zaleplon, zolpidem, and equivalents and pharmaceutically active isomer(s) and metabolite(s) thereof; and

(xiv) mood stabilizers including, for example, carbamazepine, divalproex, gabapentin, lamotrigine, lithium, olanzapine, quetiapine, valproate, valproic acid, verapamil, and equivalents and pharmaceutically active isomer(s) and metabolite(s) thereof.

In general, the administered amounts of the compound of Formula I (or a salt thereof) and the other active compound(s) are sufficient so that, when combined, they provide one or more desired therapeutic effects. Such amounts may typically be determined by one skilled in the art. For example, the amounts may, in some instances, be identified by starting with the dosages described above for the compound of Formula I (or salt thereof) and an approved or published dosage range for the other pharmaceutically active compound(s).

The compound of Formula I or a pharmaceutically acceptable salt thereof can be prepared as described herein. Various alternate reagents and changes to reaction conditions will be apparent to those skilled in the art.

It will be understood that the compound of Formula I or a salt thereof may exist in solvated (e.g., hydrated), as well as unsolvated forms. It is to be understood that the present invention encompasses all such solvated forms that possess the above-mentioned activity.

### LIST OF ABBREVIATIONS

AcOH acetic acid

DIEA diisopropylethyl amine

EtOAc ethyl acetate

Et₂O diethyl ether

NMR nuclear magnetic resonance

HPLC high performance liquid chromatography

LCMS liquid chromatography mass spectrometry

NaOAc sodium acetate

NBS N-bromosuccinimide

Sat'd aq saturated aqueous

TLC thin-layer chromatography

NOEL no observed effect level

ELISA enzyme-linked immunosorbent assay

MED minimum effective dose

s.c. subcutaneously

p.o. orally

i.p. intraperitoneally

CDS cellular dielectric spectroscopy

### EXAMPLES

The following examples are merely illustrative of embodiments of the invention, and not limiting to the remainder of this disclosure in any way.

### Example 1

### Synthesis of (R)-N*6*-ethyl-6,7-dihydro-5H-indeno[5,6-d]thiazole-2,6-diamine

The following Scheme A illustrates a method used to synthesize (R)-N*6*-ethyl-6,7-dihydro-5H-indeno[5,6-d]thiazole-2,6-diamine.

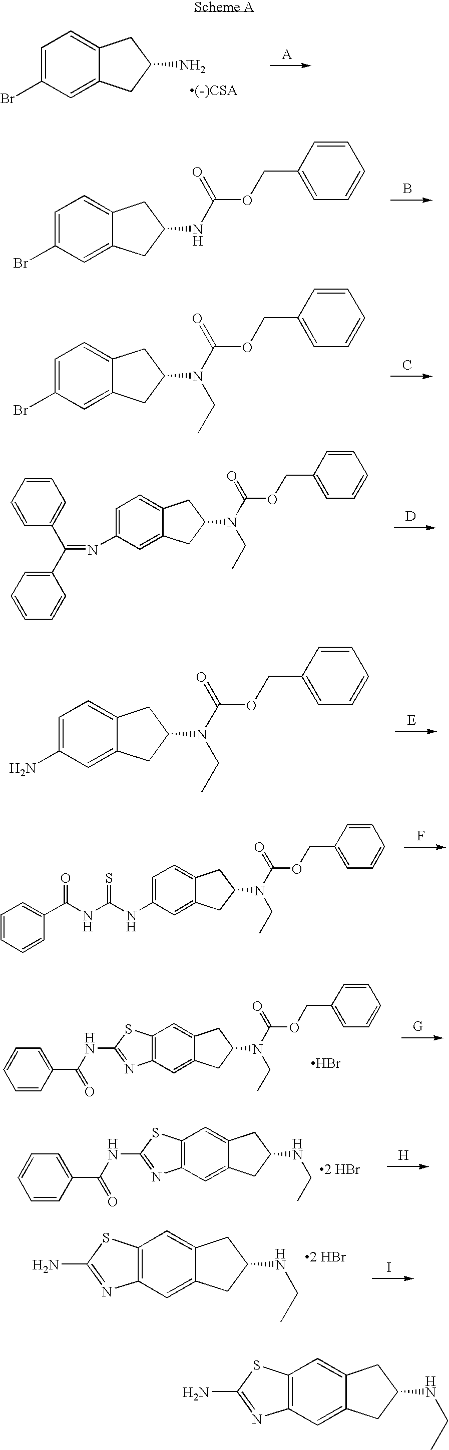

This synthesis was carried out as follows.

### Step A. Synthesis of ((S)-5-bromo-indan-2-yl)-carbamic acid benzyl ester

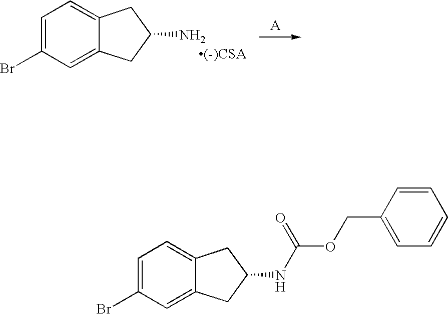

To a suspension of (S)-5-bromo-indan-2-ylamine (1R)-(−)-10-camphorsulfonate salt (109.6 g, 246.8 mmol, as prepared by the method given in Adv. Synth. Catal. 2001, 343, pp 461-472) in CH₂Cl₂ (1 L) chilled in an ice bath was added DIEA (10 mL, 617 mmol) followed by benzyl chloroformate (36.5 mL, 259 mmol) dropwise over 5-10 min. This mixture was stirred for 2 h, at which time H₂O (100 mL) was added. The phases were separated and the organic phase washed with 1M HCl (˜100 mL), H₂O (˜100 mL) and sat'd aq NaHCO₃ (˜100 mL), H₂O (˜100 mL) and sat'd aq NaCl (˜100 mL), then concentrated. The resultant yellow solid was triturated with Et₂O (˜50 mL), collected by vacuum filtration, and rinsed with Et₂O (˜10-20 mL). The resultant solid was air-dried to afford ((S)-5-bromo-indan-2-yl)-carbamic acid benzyl ester (82.6 g, 239 mmol, 97%).

¹H NMR (DMSO-d6), δ: 2.84-2.70 (m, 2H), 3.20-3.04 (m, 2H), 4.32-4.23 (m, 1H), 5.02 (s, 2H), 7.15 (d, J=7.7 Hz, 1H), 7.40-7.28 (m, 7H), 7.61-7.57 (m, 1H).

### Step B. Synthesis of ((S)-5-bromo-indan-2-yl)-ethyl-carbamic acid benzyl ester

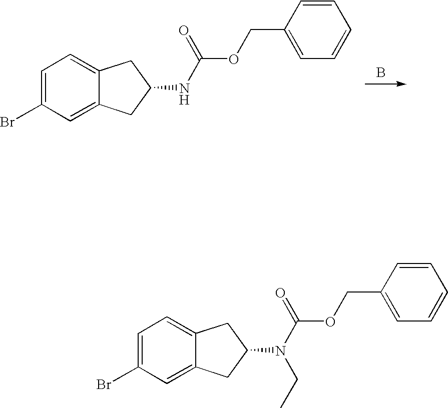

A solution of ((S)-5-bromo-indan-2-yl)-carbamic acid benzyl ester (86.1 g, 249 mmol) in dry DMF (300 mL) under N₂ was cooled in an ice bath. NaH (14.8 g, 370 mmol of a 60% dispersion in mineral oil) was added in 5 g portions, and the mixture was allowed to stir for 30 min after the last of the NaH had been added. Ethyl iodide (40.3 g, 20.4 mL, 258 mmol) was added in a fast stream over ˜1 min. The ice bath was removed and the reaction was stirred for 4 h, at which point it was again cooled in an ice bath before it was carefully quenched with H₂O, which caused gas evolution. The reaction mixture was diluted to ˜1 L with H₂O, and extracted with hexanes (3 times, with a total volume of ˜1 L). The combined organic phases were washed twice with H₂O (˜200 mL each), and filtered through filter paper. The filtrate was concentrated to a brown oil, which was purified by silica gel chromatography (0-20% EtOAc/hexanes) to afford ((S)-5-bromo-indan-2-yl)-ethyl-carbamic acid benzyl ester (86.9 g, 232 mmol, 93%).

¹H NMR (DMSO-d6), δ: 1.05 (t, J=6.9 Hz, 3H), 3.14-2.94 (m, 4H), 3.24 (q, J=7.0 Hz, 2H), 4.71-4.64 (m, 1H), 5.09 (s, 2H), 7.15 (d, J=8.4 Hz, 1H), 7.41-7.28 (m, 7H).

### Step C. Synthesis of [(S)-5-(benzhydrylidene-amino)-indan-2-yl]-ethyl-carbamic acid benzylester

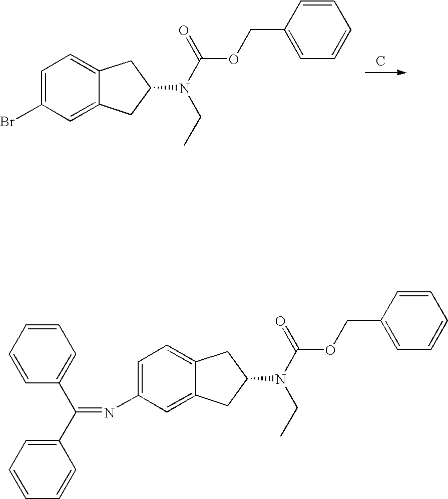

In an oven-dried 1 L three-necked flask equipped with a condenser and a thermometer was added tris(dibenzylideneacetone)dipalladium(0) (1.957 g, 2.14 mmol), 2-dicyclohexylphosphino-2′,4′,6′-triisopropylbiphenyl (2.038 g, 4.27 mmol) and toluene (150 mL) to give a brown mixture. The resulting mixture was purged with N₂ for 20 min and refluxed under N₂ for 20 min. The brown solution was allowed to cool to 60° C. Sodium tert-butoxide (16.43 g, 171.00 mmol) was added, followed by a N₂-purged (20 min) solution of ((S)-5-bromo-indan-2-yl)-ethyl-carbamic acid benzyl ester (32 g, 85.50 mmol) and redistilled benzophenone imine (17.04 g, 94.05 mmol) in toluene (80 mL) through a double-tipped needle. The temperature increased by 15° C., and the mixture became thick. The empty flask that contained the solution was rinsed with toluene (20 mL), and the rinse was added to the reaction through the double-tipped needle. The reaction mixture was stirred at 60-65° C. for 4 h. It was then allowed to cool to room temperature and filtered through a layer of diatomaceous earth. The filter-cake was rinsed with toluene (˜100 mL). The dark brownish filtrate was evaporated. The product, [(S)-5-(benzhydrylidene-amino)-indan-2-yl]-ethyl-carbamic acid benzylester, was used in the next step without further purification.

### Step D. Synthesis of ((S)-5-amino-indan-2-yl)-ethyl-carbamic acid benzyl ester

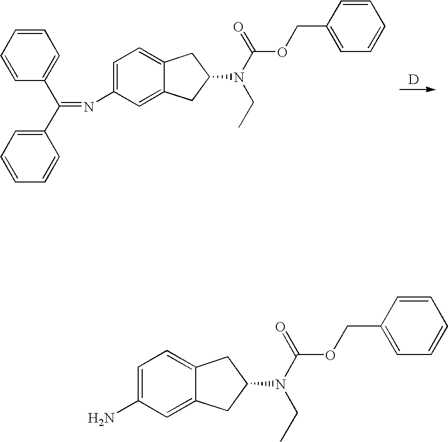

[(S)-5-(benzhydrylidene-amino)-indan-2-yl]-ethyl-carbamic acid benzylester (crude from the last step) was dissolved in methanol (350 mL) in a 1 L round-bottomed flask. Hydroxylamine hydrochloride (8.91 g, 128.25 mmol) and NaOAc (14.03 g, 171.00 mmol) were added. The resulting suspension was stirred at room temperature overnight. The suspension was filtered, and the filtrate was evaporated. The residue was stirred in CH₂Cl₂ (300 mL) for 5 min and filtered. The filter cake was rinsed with CH₂Cl₂ (50 mL) and the filtrate evaporated. The residue was purified by silica gel column chromatography (0-30% EtOAc/hexanes) to afford ((S)-5-amino-indan-2-yl)-ethyl-carbamic acid benzyl ester (24.95 g, 80.38 mmol, 94%) as a golden-colored oil.

¹H NMR (DMSO-d6), δ: 1.04 (t, J=7 Hz, 3H), 2.79-2.93 (m, 4H), 3.21 (q, J=7 Hz, 2H), 4.66 (p, J=8 Hz, 1H), 4.79 (s, br, 2H), 5.09 (s, 2H), 6.36 (dd, J=2, 8 Hz, 1H), 6.42 (s, 1H), 6.83 (d, J=8 Hz, 1H), 7.29-7.38 (m, 5H).

### Steps E and F. Synthesis of ((R)-2-benzoylamino-6,7-dihydro-5H-indeno[5,6-d]thiazol-6-yl)-ethyl-carbamic acid benzyl ester.HBr

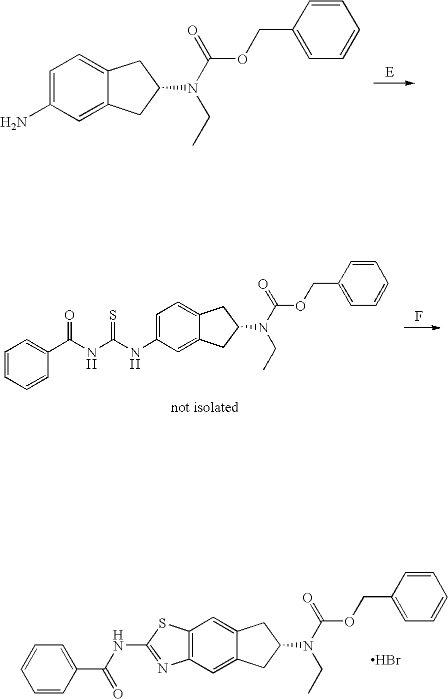

In a 2 L three-necked flask was charged a solution of ((S)-5-amino-indan-2-yl)-ethyl-carbamic acid benzyl ester (36.26 g, 116.82 mmol) in CH₂Cl₂ (300 mL). A solution of benzoyl isothiocyanate (19.64 g, 120.33 mmol) in CH₂Cl₂ (100 mL) was added and the internal temperature increased from 17° C. to 32° C. The resulting light-brownish solution was stirred for 1.5 h. The completion of the reaction was confirmed by TLC (silica gel eluted with 25% EtOAc/hexanes). A solution of NBS (21.42 g, 120.33 mmol) in CH₂Cl₂ (700 mL) was added over 10 min. The temperature increased from 19° C. to 25° C. The stirring was continued for an additional 30 min. CH₃CN (500 mL) was added, and the solution was evaporated to about 400 mL. CH₃CN (400 mL) was added. The solid was filtered, washed with CH₃CN (200 mL), and vacuum dried until no solvent dripped. The wet solid was dissolved in CH₂Cl₂ (600 mL). CH₃CN (500 mL) was added, and the resulting solution was evaporated to about 400 mL. CH₃CN (200 mL) was again added and the solid was filtered, washed with CH₃CN (200 mL), and dried under vacuum to give ((R)-2-benzoylamino-6,7-dihydro-5H-indeno[5,6-d]thiazol-6-yl)-ethyl-carbamic acid benzyl ester.HBr (35 g, 63.35 mmol, 54%) as a light yellow solid.

¹H NMR (DMSO-d6), δ: 1.08 (t, J=7 Hz, 3H), 3.11-3.23 (m, 4H), 3.28 (q, J=7 Hz, 2H), 4.78 (p, J=8 Hz, 1H), 5.11 (s, 2H), 7.27-7.40 (m, 5H), 7.56 (t, J=7.75 Hz, 2H), 7.61 (s, 1H), 7.66 (t, J=7.25 Hz, 1H), 7.81 (s, 1H), 8.13 (d, J=8 Hz), 12.76 (s, br, 1H, HBr)

### Step G. Synthesis of N-((R)-6-ethylamino-6,7-dihydro-5H-indeno[5,6-d]thiazol-2-yl)-benzamide.2HBr

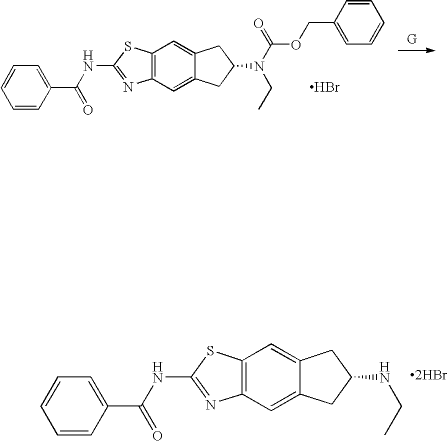

A 1 L round-bottomed flask was charged hydrobromic acid (33% in AcOH, 500 mL) and triisopropylsilane (34.5 mL, 168.41 mmol). The mixture was stirred and ((R)-2-benzoylamino-6,7-dihydro-5H-indeno[5,6-d]thiazol-6-yl)-ethyl-carbamic acid benzyl ester.HBr (34.5 g, 62.45 mmol) was added. The resulting suspension was stirred at room temperature for 2 h. The reaction mixture was evaporated to about 100 mL. Et₂O (500 mL) was added, and the solid was filtered, washed with fresh Et₂O (250 mL), and dried. 31.07 g (62.23 mmol, 99.6%) of N-((R)-6-ethylamino-6,7-dihydro-5H-indeno[5,6-d]thiazol-2-yl)-benzamide.2HBr was obtained as an off-white solid.

¹H NMR (DMSO-d6), δ: 1.24 (t, J=7.25 Hz, 3H), 3.05-3.10 (m, 2H), 3.14-3.20 (m, 2H), 3.39-3.45 (m, 2H), 4.10 (p, J=6.75 Hz, 1H), 7.57 (t, J=7.75 Hz, 2H), 7.66 (d, J=7.5 Hz, 1H), 7.68 (s, 1H), 7.91 (s, 1H), 8.13 (d, J=7.75 Hz, 2H), 8.69 (s, br, 2H), 12.79 (s, br, 1H, HBr)

### Step H. Synthesis of (R)-N*6*-ethyl-6,7-dihydro-5H-indeno[5,6-d]thiazole-2,6-diamine.2HBr

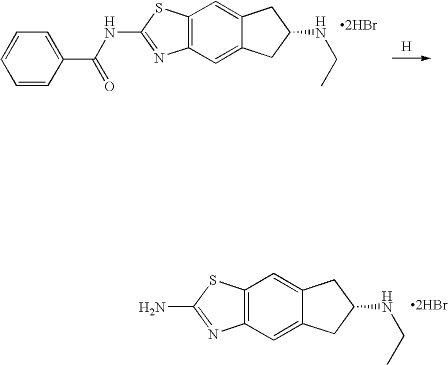

A 2 L round-bottomed flask was charged with N-((R)-6-ethylamino-6,7-dihydro-5H-indeno[5,6-d]thiazol-2-yl)-benzamide.2HBr (31.0 g, 62.09 mmol) and 48% hydrobromic acid (500 mL) to give a suspension. The reaction mixture was heated to reflux and became a clear solution after 1.5 h. The completion of the reaction was confirmed by disappearance of starting material as monitored by LCMS after 6 hr of reflux. The volatiles were removed under reduced pressure. The residue was stirred in CH₃CN (1 L) for 10 min, filtered, and washed with fresh CH₃CN (250 mL). The product was dried under vacuum to give 24.9 g (63.01 mmol, 101%) of (R)-N*6*-ethyl-6,7-dihydro-5H-indeno[5,6-d]thiazole-2,6-diamine.2HBr as an off-white solid.

¹H NMR (DMSO-d6), δ: 1.23 (t, J=7.25 Hz, 3H), 3.00-3.06 (m, 2H), 3.08-3.14 (m, 2H), 3.31-3.38 (m, 2H), 4.06 (p, J=7 Hz, 1H), 7.35 (s, 1H), 7.71 (s, 1H), 8.74 (s, br, 3H), 8.92 (s, br, 2H)

### Step I. Synthesis of (R)-N*6*-ethyl-6,7-dihydro-5H-indeno[5,6-d]thiazole-2,6-diamine

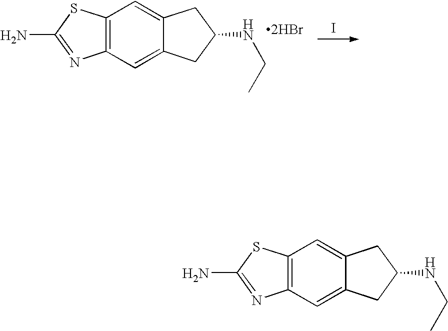

In a 2 L round-bottomed flask was dissolved (R)-N6-ethyl-6,7-dihydro-5H-indeno[5,6-d]thiazole-2,6-diamine.2HBr (54.2 g, 137.12 mmol) in H₂O (800 mL) to give a light yellow solution. The solution was filtered through a 1.0 μm GMF-150 syringe filter. 2.5 N aq NaOH (121 mL, 301.66 mmol) was added over 10 min. The precipitated solid was filtered and washed with H₂O (˜600 mL) until the pH of the washing reached 6.5. The solid was dried under vacuum to give 30.5 g (130.71 mmol, 95%) of (R)-N*6*-ethyl-6,7-dihydro-5H-indeno[5,6-d]thiazole-2,6-diamine as an off-white solid.

¹H NMR (DMSO-d6), δ: 1.02 (t, J=7 Hz, 3H), 2.58-2.67 (m, 4H), 3.05 (dt, J=6.5, 15.5 Hz, 2H), 3.51 (p, J=7 Hz, 1H), 7.14 (s, 1H), 7.21 (s, 2H), 7.40 (s, 1H).

### Example 2

### Scale-Up of (R)-N*6*-ethyl-6,7-dihydro-5H-indeno[5,6-d]thiazole-2,6-diamine Synthesis

Reaction Scheme A was scaled up approximately ten-fold. The scaled-up reaction was carried out similarly to the manner in which Reaction Scheme A was carried out above except for scale and the following differences:

In the scaled up version of the method in Step A, the solvent was changed from CH₂Cl₂ to acetonitrile. Doing so allowed product isolation by addition of water and subsequent filtration of the product. The scaled up method benefits from the lack of extractive workup and the lack of halogenated solvents.

In the scaled up method, it was determined that it was not necessary to distill the benzophenone imine in Step C. Thus, the distillation was omitted without compromising imine formation.

In Steps E and F in the scaled up method, the crude hydrolysis product was dissolved in toluene rather than CH₂Cl₂. The by-product was filtered off, and the toluene filtrate purified by silica gel chromatography (first EtOAc (1-3%)/toluene, and then EtOAc (5-25%)/hexane).

In the scaled up method, purification of Step G was achieved by suspending the solid (1168 g) in CH₃CN (10 volumes, 12 L) and CH₂Cl₂ (2 volumes, 2 L). The suspension was refluxed for 2 h and 3 L of solvent was removed by distillation over 2 h. The distillate was cooled overnight. The solid was collected by filtration and dried. The process was repeated a second time to complete the purification of the final product.

In the neutralization step (Step I) of the scaled up method, the di-HBr salt (756.6 g) was dissolved in 10 L of distilled water and filtered through diatomaceous earth. This solution was added dropwise over 2 h to 5 L of 1N KOH solution and stirred an additional 2 h. Solid was collected by filtration and washed 4 times with distilled water, and then vacuum dried to afford the desired product at a yield of about 82%.

### Example 3

### In Vitro Assay Procedures

Affinity (Ki) to the D2 receptor was measured with an [³H]-raclopride binding assay. D2 antagonism (IC₅₀) was measured with a GTPγS assay. The GTPγS assay, however, failed to detect agonism of aripiprazole in our hands and others (Jordon S, et. al., “Dopamine D2 receptor partial agonists display differential or contrasting characteristics in membrane and cell-based assays of dopamine D2 receptor signaling,” Prog Neuropsychopharmacol Biol Psychiatry, 31 (2):348-56 (Mar. 30, 2007); Epub Oct. 27, 2006). This highlights the limitations of using a GTPγS assay to identify D2 partial agonists. Assays measuring a more downstream level of cell signaling, such as cAMP and extracellular impedance across a cell layer, tend to be more sensitive to detect agonism to provide reliable agonism of D2 partial agonists. Changes in extracellular impedance across a cell layer can be measured by a cellular dielectric spectroscopy (CDS) with a CellKey instrument. We observed that data of D2 agonists from the CDS assay correlated well with those from the cAMP assay, with less variation. Thus, CDS was used to measure the agonist activity of partial D2 agonists. The maximum agonism effect (Emax) was relative to the maximum effect of dopamine. The following discussion provides a description of the assays and results.

### In Vitro Assay Procedures

CHO—K1cells stably transfected with the dopamine D2s receptor were used in the experiments and maintained in Ham's F12 culture medium supplemented with 2 mM L-glutamine, 10% FBS, and 500 μg/ml Hygromycin.

### D2 Receptor Binding Assay

The ability of test compounds to displace ³H-raclopride at the D2S receptor was determined on membranes from D2s-transfected CHO cells (Bmax 13 pmol/mg protein). An assay uses a standard 96-well glass fiber filter plate to retain radioligand bound by the receptor. Retained ³H is determined in a TopCount scintillation plate counter following the addition of a liquid scintillant to each well. Compounds are evaluated for their potency using competition curve analysis, resulting in calculated Ki values.

### D2 Receptor In Vitro Functional Assays

GTPγS assay was performed substantially as described by Lazareno. (Lazareno, S., (1999) Measurement of agonist-stimulated [³⁵S]-GTPγS binding to cell membranes. Methods in Molecular Biology 106: 231-245). Antagonist activity of compounds was determined by the ability of test compounds to block dopamine-stimulated [³⁵S]-GTPγS binding to cell membranes from D2s stably-transfected CHO cells. This assay, however, is not very sensitive to agonist activity. Consequently, another, more sensitive, technique was used.

Cellular dielectric spectroscopy (CDS) was used to measure the agonist activity of partial D2 agonists with a CellKey instrument. A CellKey instrument measures changes in extracellular impedance across a cell layer. In this assay, an increase in impedance (positive dZiec value) for this receptor indicates an agonist effect. Compounds were evaluated for their potency and efficacy/intrinsic activity using dose response curve analysis, resulting in EC₅₀ and Emax (curve top) values. Specificity of the cellular response elicited by the compounds was determined by testing them on cells which have been preincubated with 1 μM raclopride, a silent D2-specific antagonist that will block the downstream effects mediated by the D2 receptor and is identical to buffer baseline when tested alone in the assay. The protocol is generally described in Peters, M. F. et al, “Evaluation of Cellular Dielectric Spectroscopy, a Whole-Cell, Label-Free Technology for Drug Discovery on Gi-Coupled GPCRs,” J Biomol Screen April 2007; 12(3):312-9. Epub Feb. 16, 2007. doi:10.1177/1087057106298637.

### Results

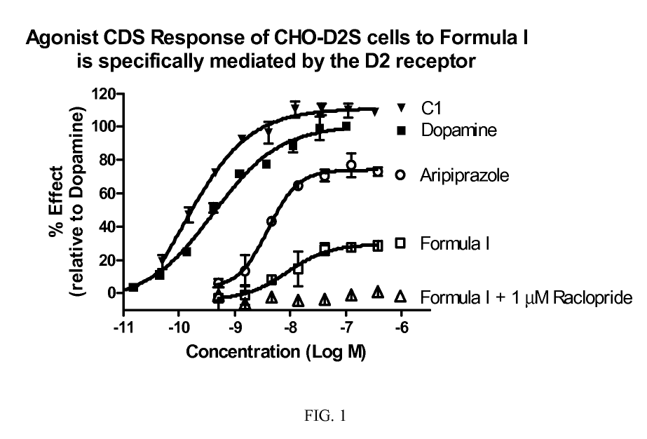

The results were unexpected in that the properties of the compound of Formula I were different, in terms of partial agonism, to its enantiomer (C1) as well as other structural analogs. The results are shown in FIG. 1 and Table 1 (mean values±SD).

TABLE 1
In Vitro Assay Results

| | Stereochemical | | Antagonism | Agonism |
|---|---|---|---|---|
| | designation of the | | (GTPγS) | (CDS) |
| Compound | lone stereocenter | pKi (M) | pIC₅₀ (M) | Emax |
| 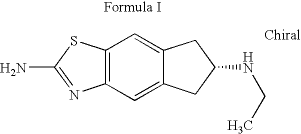 | R | 6.83 ± 0.31 | 6.15 ± 0.31 | 21 ± 1.6 |
| 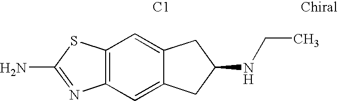 | S | 7.04 ± 0.18 | <4.92 ± 0.9 | 110 ± 7.3 |
| 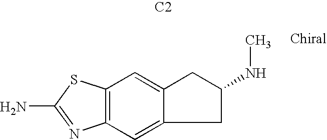 | R | 6.82 ± 0.11 | 6.38 ± 0.01 | NA* |
| 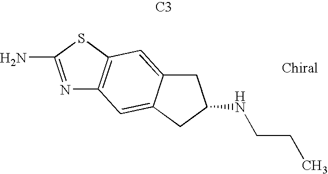 | R | 6.94 ± 0.04 | 6.36 ± 0.04 | NA* |
| 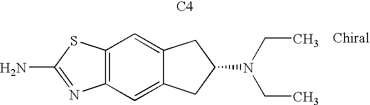 | R | 7.66 ± 0.20 | 7.13 ± 0.08 | NA* |
| Aripiprazole | | 9.17 ± 0.49 | 8.09 ± 0.30 | 69 ± 1.1 |
| Haloperidol | | 9.94 ± 0.20 | 8.29 ± 0.16 | NA* |
| Risperidone | | 8.78 ± 0.50 | 7.98 ± 0.27 | NA* |

NA*: Not active in the assay (below the assay variation 3 × SD).

In addition, work was done with the CDS assay to demonstrate that the D2 partial agonism exhibited by the compound of Formula I is specifically blocked by D2 antagonist raclopride, demonstrating D2-mediated response of the compound of Formula I.

### Example 4

### In Vivo Assay Experimental Test Procedures

Additional studies in vivo supported the above-discussed in vitro effects suggesting this compound for use as an antipsychotic and differentiating it from its (S)-enantiomer and other structurally similar compounds.

### D-Amphetamine-Induced Hyperlocomotor Activity (LMA) in Habituated Rat Model

LMA was assessed in male Long Evans rats using a paradigm that included a habituation phase followed by administration of 1 mg/kg D-amphetamine. Animals were allowed to acclimatize to the testing room for 1 hour before being weighed and placed into activity chambers. Thirty minutes after LMA measurements began, animals were briefly removed, subcutaneously dosed with vehicle or test drug at different doses (μmol/kg), and returned to the chambers. After 30 minutes, animals were again removed and dosed with vehicle or D-amphetamine at 1 mg/kg (s.c.). After returning animals to the activity chambers, LMA was assessed for 60 minutes. Haloperidol (0.1 mg/kg dissolved in H₂O) was subcutaneously administered 15 minutes before D-amphetamine. Statistical analysis was made of total distance traveled after D-amphetamine administration using ANOVA and Tukey's post hoc analysis where appropriate. All values are expressed as Mean and SD.

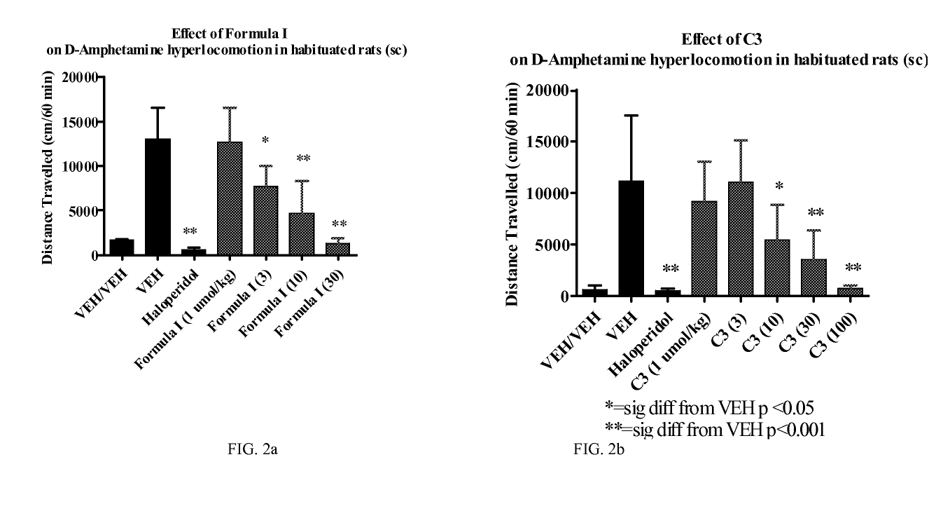

Antipsychotics lead to reversal of LMA. The compound of Formula I was found to be active (MED 3 μmol/kg) in this assay, as was C3 (MED 10 μmol/kg), further supporting the observed in vitro D2 antagonism and use as an antipsychotic. FIGS. 2a and 2b show the effect of the compound of Formula I and compound C3 on D-amphetamine hyperlocomotion in habituated rats.

### Conditioned Avoidance Responding (CAR) Assay

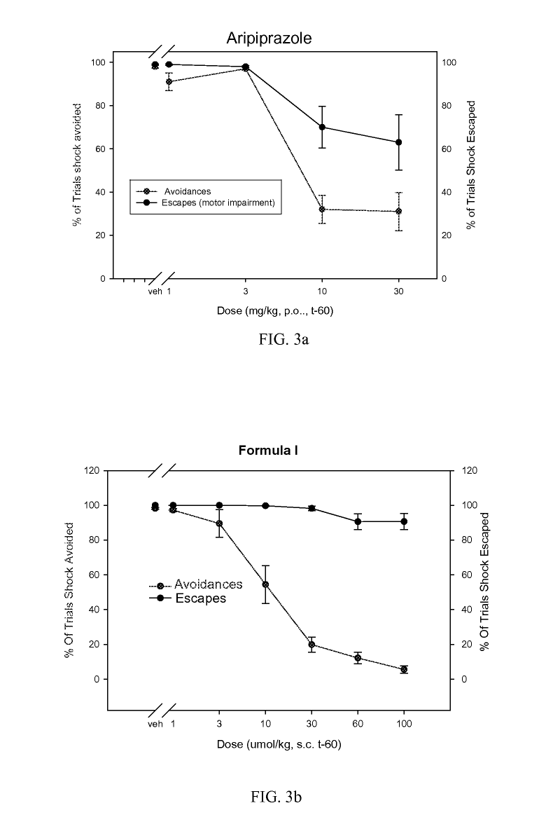

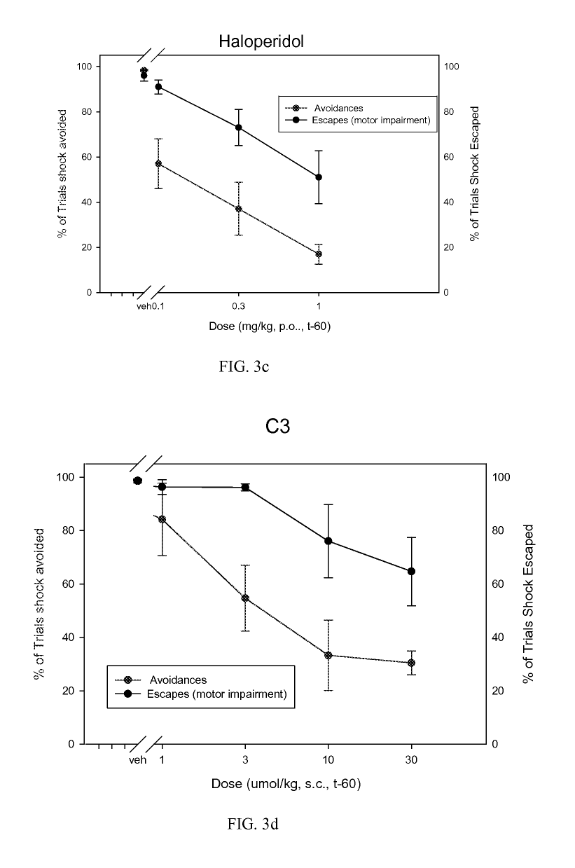

Male Long-Evans rats were trained to traverse to the opposite side of a standard shuttle cage following presentation of an auditory and visual stimulus in order to avoid delivery of electric shock to the floor of the cage. Daily sessions consisted of up to 80 trials. If shock was delivered, animals always had the opportunity to escape the shock by traversing to the opposite side of the cage. Drug was administered (via s.c. or p.o. route) 60 min prior to testing and the percentage of trials in which shock was avoided and escaped was recorded. FIGS. 3a, 3b, 3c, and 3d show data for the compound of Formula I, comparative compound C3, and two known antipsychotics.

The CAR assay is sensitive to antipsychotics (D2 antagonists). The compound of Formula I was effective in this antipsychotic animal model (as measured by shock avoidance) without motor impairment (as measured by shock escapes) up to 100 μmol/kg. In contrast, comparative compound C3 and the other antipsychotics, such as haloperidol, and aripiprazole exhibited motor impairment though effective in the animal model. When comparing the D2 selective compounds of Formula I and C3, the results suggest that the partial D2 agonism of Formula I mitigated motor impairment. Aripiprazole also exhibited motor impairment, most likely due to its non-D2 pharmacological activities.

### Catalepsy Assay

CF-1 male mice or Sprague Dawley rats are dosed (via i.p., p.o. or s.c. route) with a test compound at given concentrations or a vehicle. For the positive control, one group of mice is always dosed with 2 mg/kg of haloperidol s.c. At 60 min. and 4 hr. after dosing, the experimenter gently places both forepaws of each animal on a metal bar (4 mm in diameter) that is fixed horizontally 5 cm above the test floor. The length of time (in seconds) during which each mouse maintains the initial forepaw bar position is recorded (cataleptic pose). Maximum cut-off observation time is 60 seconds. Results are expressed as means (in sec) for each dose group.

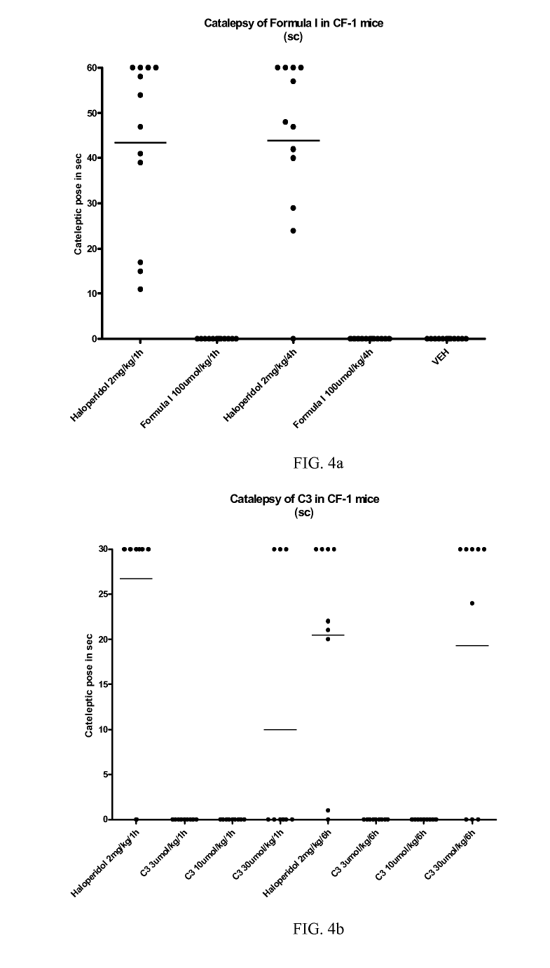

EPS is a common side effect, believed to be D2-mediated, for some marketed antipsychotics. Catalepsy is a condition characterized by muscular rigidity, as well as fixity of posture and is used as an animal model to predict akinesia and rigidity aspects of human EPS. Haloperidol, a typical antipsychotic D2 antagonist with high risk of EPS incidence in patients, induced catalepsy in rats and mice. The compound of Formula I does not show catalepsy in rats or mice up to 100 μmol/kg, much higher than those that lead to efficacy in the LMA or CAR assays. C3 exhibited catalepsy when it was administered at 30 μmol/kg in mice. Thus, these results suggest that partial D2 agonism of Formula I mitigates catalepsy, which, in turn, suggests EPS as well. FIGS. 4a and 4b show results of the mouse catalepsy assay.

### Prolactin Assay

As discussed above, hyperprolactinemia is a side effect that can be observed following administration of D2 antagonists. In contrast, administration of D2 agonists to human subjects results in large reductions in prolactin levels in blood (hypoprolactinemia). Potent D2 antagonists, such as risperidone, can lead to a large elevation of prolactin in blood of rodent and man. In humans, use of less potent antagonists such as clozapine or quetiapine results in small, transient increases in prolactin which generally do not have significant clinical impact. Administration of the partial agonist aripiprazole leads to a small increase in blood prolactin in the rat but a small decrease in man. Due to the apparent lack of correlation between the rat and human, seen in our hands and in the literature, the inventors are not convinced that the rat prolactin assay is entirely predictive of the effect in humans. Nevertheless, the assay was run to assess the effect Formula I and reference compounds on prolactin levels in rat.

Male Sprague Dawley rats were subcutaneously administered with vehicle or test compounds. Trunk blood was collected one-hour post dose and plasma evaluated by an ELISA assay to determine prolactin levels.

In our tests, all compounds tested had significant effects on rat plasma prolactin levels. Risperidone was the most potent, with a no observable effect level (NOEL) of 0.07 μmol/kg. At a dose of 0.2 μmol/kg risperidone administration produced reliable hyperprolactinemia and this dose was therefore used as a positive control across experiments. A NOEL of 2.2 μmol/kg was determined for aripiprazole. Formula I had a NOEL of 3 μmol/kg. C3 had a NOEL of 10 μmol/kg under the conditions of these experiments.

### Example 5

### Determination of the Absolute Configuration of Compound of Formula I

To a sample of (R)-N*6*-ethyl-6,7-dihydro-5H-indeno[5,6-d]thiazole-2,6-diamine (ca 100 mgs) was added a small volume of methanol. The volume was just enough to dissolve the sample. An approximately equal volume of methyl tert-butyl ether was added. This solution was lightly covered and allowed to slowly evaporate. This afforded crystals that were utilized in the single crystal x-ray analysis. Colorless needle crystals were obtained and used “as is”. The diffraction data were collected on the OXFORD Xcalibur3 diffractometer at John Hopkins University, and the crystal structure was solved and refined with the SHELXTL software package. The data is shown in Tables 2 and 3 below.

TABLE 2
Crystal data and structure refinement for compound of Formula I

| | | |
|---|---|---|
| Empirical formula | C12H15N3S1 | |
| Temperature | 293(2) K | |
| Wavelength | 0.71073 Å | |
| Crystal system | Orthorhombic | |
| Space group | P2(1)2(1)2(1) | |
| Unit cell dimensions | a = 6.9578(2) Å | α = 90° |
| | b = 7.3442(2) Å | β = 90° |
| | c = 25.6749(6) Å | γ = 90°. |

| | |
|---|---|
| Volume | 1311.97(6) Å³ |
| Z | 4 |
| Density (calculated) | 1.364 Mg/m³ |
| Absorption coefficient | 0.246 mm⁻¹ |
| F(000) | 576 |
| Crystal size | ? x ? x ? mm³ |
| Theta range for data collected | 4.03 to 29.54° |
| Index ranges | −9 <= h <= 8, −9 <= k <= 9, |
| | −34 <= l <= 34 |
| Reflections collected | 15365 |
| Independent reflections | 3396 [R(int) = 0.0381] |
| Completeness to theta = 29.54° | 94.7% |
| Absorption correction | none |
| Refinement method | Full-matrix least-squares on F² |
| Data/restraints/parameters | 3396/0/168 |
| Goodness-of-fit on F² | 1.078 |
| Final R indices [I > 2sigma(I)] | R1 = 0.0500, wR2 = 0.1301 |
| R indices (all data) | R1 = 0.0583, wR2 = 0.1348 |
| Absolute structure parameter | 0.01(11) |
| Largest diff. peak and hole | 1.027 and −0.476 e · Å⁻³ |

TABLE 3
Bond lengths [Å] and angles [°] for compound of Formula I

| | |
|---|---|
| C(1)—N(1) | 1.306(3) |
| C(1)—N(2) | 1.343(3) |
| C(1)—S(1) | 1.755(3) |
| C(2)—N(1) | 1.394(3) |
| C(2)—C(3) | 1.396(4) |
| C(2)—C(10) | 1.416(3) |
| C(3)—C(4) | 1.384(3) |
| C(4)—C(8) | 1.398(3) |
| C(4)—C(5) | 1.510(4) |
| C(5)—C(6) | 1.539(3) |
| C(6)—N(3) | 1.471(3) |
| C(6)—C(7) | 1.545(4) |
| C(7)—C(8) | 1.508(3) |
| C(8)—C(9) | 1.388(3) |
| C(9)—C(10) | 1.388(3) |
| C(10)—S(1) | 1.740(2) |
| C(11)—N(3) | 1.475(4) |
| C(11)—C(12) | 1.511(4) |
| N(1)—C(1)—N(2) | 125.6(2) |
| N(1)—C(1)—S(1) | 116.10(19) |
| N(2)—C(1)—S(1) | 118.3(2) |
| N(1)—C(2)—C(3) | 125.4(2) |
| N(1)—C(2)—C(10) | 115.1(2) |
| C(3)—C(2)—C(10) | 119.5(2) |
| C(4)—C(3)—C(2) | 118.3(2) |
| C(3)—C(4)—C(8) | 121.5(2) |
| C(3)—C(4)—C(5) | 128.2(2) |
| C(8)—C(4)—C(5) | 110.2(2) |
| C(4)—C(5)—C(6) | 103.0(2) |
| N(3)—C(6)—C(5) | 113.6(2) |
| N(3)—C(6)—C(7) | 111.7(2) |
| C(5)—C(6)—C(7) | 104.4(2) |
| C(8)—C(7)—C(6) | 103.0(2) |
| C(9)—C(8)—C(4) | 121.4(2) |
| C(9)—C(8)—C(7) | 128.4(2) |
| C(4)—C(8)—C(7) | 110.1(2) |
| C(8)—C(9)—C(10) | 117.1(2) |
| C(9)—C(10)—C(2) | 122.2(2) |
| C(9)—C(10)—S(1) | 128.50(19) |
| C(2)—C(10)—S(1) | 109.25(18) |
| N(3)—C(11)—C(12) | 110.8(2) |
| C(1)—N(1)—C(2) | 110.4(2) |
| C(6)—N(3)—C(11) | 112.1(2) |
| C(10)—S(1)—C(1) | 89.09(12) |

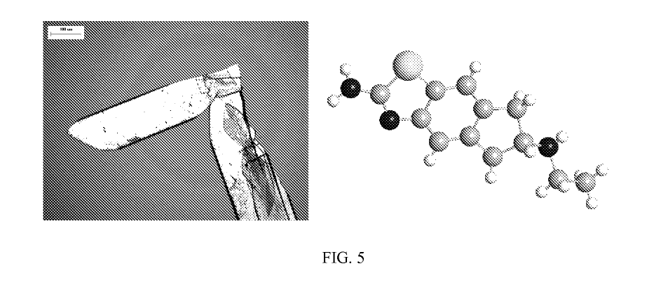

The absolute configuration of the molecule was established by using the anomalous dispersions of the S atom in the molecule. The molecule was found to be (R)-isomer (Absolute structure parameter is 0.01(11)). See FIG. 5.

### Example 6

### Alternative Synthesis of (R)-N*6*-ethyl-6,7-dihydro-5H-indeno[5,6-d]thiazole-2,6-diamine

The following Scheme B illustrates an alternative method used to synthesize (R)-N*6*-ethyl-6,7-dihydro-5H-indeno[5,6-d]thiazole-2,6-diamine.

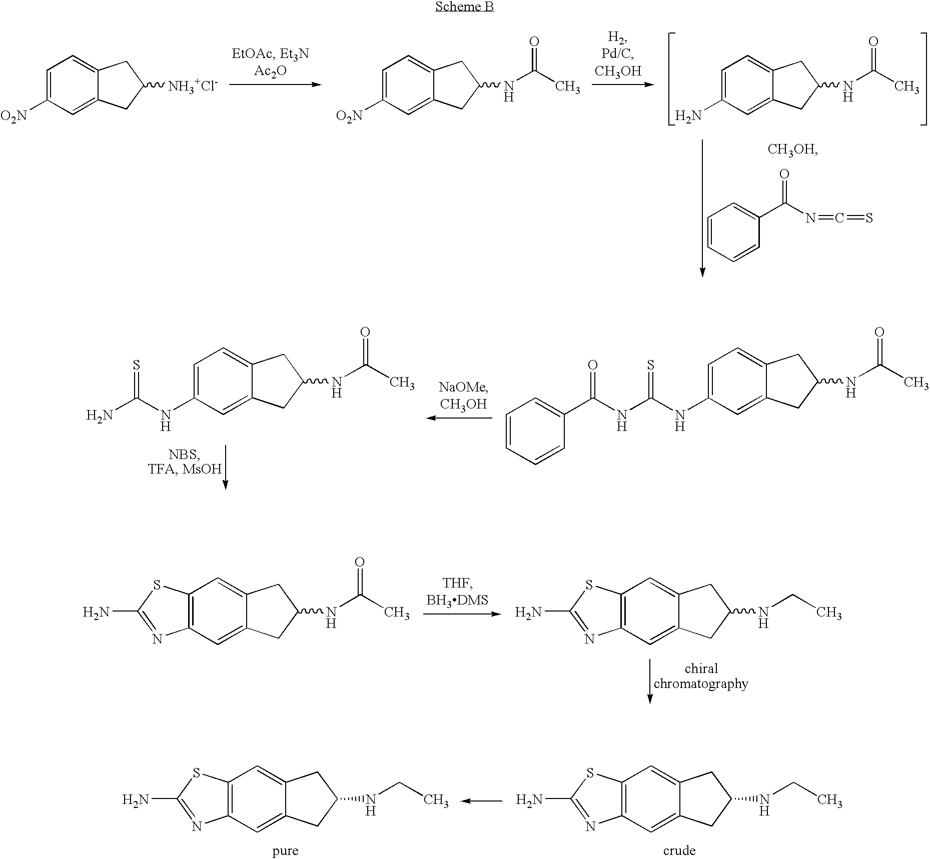

This synthesis was carried out as follows.

### Step A. Synthesis of N-(2,3-dihydro-5-nitro-1H-inden-2-yl)acetamide

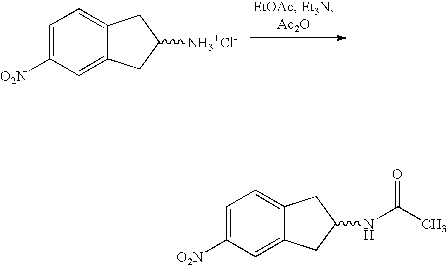

2-Amino-5-nitroindane-HCl (33 kg, 160 moles), ethyl acetate (240 kg, 2270 moles), and triethylamine (31 kg, 310 moles) were charged to reactor at 18-25° C. The resulting mixture was stirred at this temperature range for ≧15 minutes. Acetic anhydride (18 kg, 180 moles) was dissolved in ethyl acetate (60 kg, 680 moles), and then dosed to the reactor over a period of ≧30 minutes at 18-30° C. Afterward, the mixture was stirred for ≧30 minutes at 18-30° C. An IP sample was collected for analysis.

Process grade 1 water (67 kg, 3970 moles) was charged to the reactor. The mixture was stirred for ≧15 minutes at 18-30° C. The agitator was then turned off for ≧15 minutes to allow the phases to separate. The lower aqueous phase was removed and discarded. Ethyl acetate was then distilled off at about 77° C. and atmospheric pressure until a volume of 130±10 L was obtained. Heptanes (114 kg, 1140 moles) were dosed over a period of ≧2 hours at 50-70° C. The product started to precipitate almost immediately when the dosing started. The crystal suspension was cooled over a period of ≧1 hour to 18-25° C., and then stirred for ≧30 minutes. The crystal suspension was filtered in a centrifuge. There were still lumps of product left in the reactor, so the mother liquor was re-circulated back to the reactor and centrifuged again. The filtered product was washed with heptanes (57 kg, 570 moles). The resulting product was dried in a vacuum tray dryer at 65° C. A sample was collected after 8 hours of drying to check for residual solvent. The product was packed in fiber drums with double PE-bag liner and sampled for analysis. A total of 62.4 kg of product was isolated after drying.

### Step B. Synthesis of N-(2,3-dihydro-5-(phenylmethanonylthiourea-3-yl)-1H-inden-2-yl)acetamide

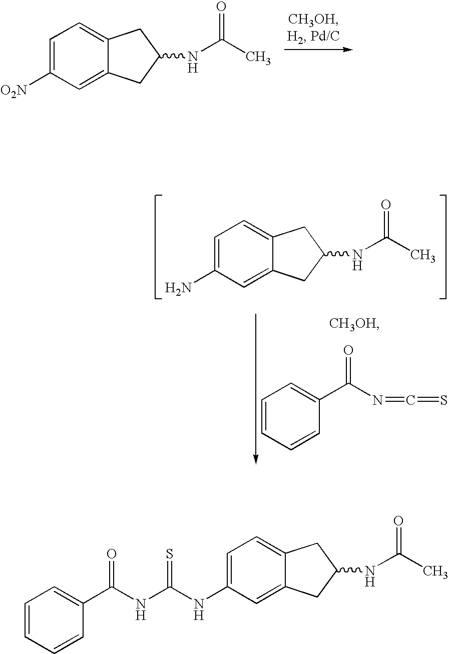

N-(2,3-dihydro-5-nitro-1H-inden-2-yl)acetamide (31 kg, 140.6 moles) and methanol (380 L, 9943 moles) were charged to a reactor and stirred for ≧15 minutes at 25-30° C. until the N-(2,3-dihydro-5-nitro-1H-inden-2-yl)acetamide dissolved. The resulting solution was transferred to a second reactor containing 3% Pd/C catalyst (2.5 kg). After rinsing the transfer pipe with methanol (31 L, 764 moles), the reduction reaction was initiated by increasing the agitation of the mixture at a temperature of 20-30° C. and pressure of 3.0-3.5 bars in a H₂ atmosphere. These conditions were continued until the solution stopped consuming hydrogen. A sample was collected for analysis.

The catalyst was filtered off. The filter was washed with methanol, which, in turn, was returned to the filtered mixture. Benzylisothiocyanate (22.5 kg, 138 moles) was then dosed over a period of ≧30 minutes at 18-30° C. The glass container containing the benzylisothiocyanate was rinsed with methanol (5 L, 159 moles), which, in turn, also was added to the reactor. The resulting mixture was stirred for 1-2 hours at 20-30° C., which afforded a crystal suspension. A sample was collected for analysis. The crystal suspension was filtered via centrifugation. The filtered product was washed with methanol (31 L, 758 moles). The mother liquor and washing liquid were discarded.

### Step C. Synthesis of 1-(2-acetamido-2,3-dihydro-1H-inden-6-yl)thiourea

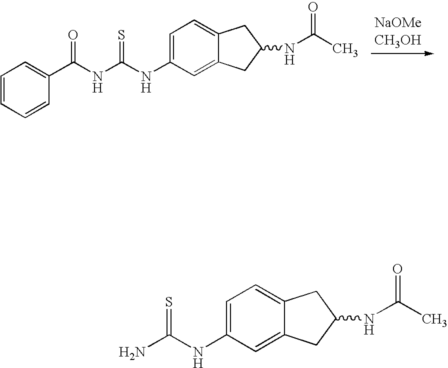

N-(2,3-dihydro-5-(phenylmethanonylthiourea-3-yl)-1H-inden-2-yl)acetamide (53 kg, 123.1 moles) and methanol (436 L, 10772 moles) were charged to a reactor and stirred for ≧30 minutes at 25-35° C. Thirty percent sodium methoxide (NaOMe, 25 kg, 141.5 moles) in methanol and additional methanol (10 kg, 313.1 moles) was then charged. The resulting solution was stirred for ≧30 minutes at 25-35° C. A sample was collected for analysis.

Process grade 1 water (220 L, 12222 moles) was charged to the reactor at 10-35° C. Methanol was then distilled off under vacuum at ≦50° C. until the desired volume was collected (520 L). Process grade 1 water (90 L, 5015 moles) and acetic acid (1.5 kg, 16.7 moles) were charged to achieve a pH of 7-9. The resulting mixture was then stirred for ≧30 minutes at 25-35° C. The resulting crystal suspension was filtered via centrifugation. The filtered product was washed with process grade 1 water (90 L, 5015.3 moles). The mother liquor and washing liquid were discarded. The product was dried using a vacuum tumble dryer at 70° C. until LOD≦1.0%. The product was packed in fiber drums with double PE-bag liner and sampled for analysis. A total amount of 62.4 kg of dry product was isolated after drying.

### Step D. Synthesis of 2-amino-6-acetylamino-6,7-dihydro-5H-indeno[5,6-d]thiazole

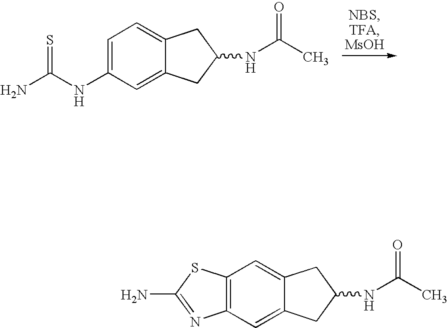

Trifluoroacetic acid (316.48 mL, 4.19 moles) was charged to a 2 L jacketted reactor fitted with a temperature probe, reflux condensor, overhead stirrer, N₂ inlet, and 250 ml dropping funnel. The trifluoroacetic acid was cooled to 11-15° C. while stirring. Afterward, 1-(2-acetamido-2,3-dihydro-1H-inden-6-yl)thiourea (86 g, 317.3 mmoles) was added over a 7-minute period. After stabilizing the temperature of the mixture at 11-15° C., methane sulfonic acid (“MsOH,” 79.12 mL, 1.21 moles) was added over a 3-minute period. After the temperature of the mixture was stabilized 15-18° C., a solution of N-bromosuccinimide (“NBS,” 56.48 g, 317.3 mmoles) and trifluoroacetic acid (118.7 mL, 1.57 moles) was added over a period of >1 hour. The transfer line was washed with trifluoroacetic acid (39.56 mL, 523.2 moles), which also was added to the mixture. The mixture was then maintained at 20° C. for 1.5 hours. Afterward, a sample was collected for analysis.

Trifluoroacetic acid was removed by distillation under vacuum (250 mbar reduced gradually to 150 mbar with the jacket temperature set at 95° C.) until 2.6 rel vols remained. The temperature of the mixture was then cooled to 20° C., and the vacuum was released. Acetonitrile (237.4 mL, 4.53 moles) was added over a 3-minute period. After cooling the mixture to 5-15° C., water (158.24 mL, 8.78 moles) was added over a 15-minute period. The jacket temperature was set at 20° C., and then ammonium hydroxide (roughly 60 g, 0.6 moles) was slowly added over a period of 30-60 minutes. After an additional 30-60 minutes, additional ammonium hydroxide (roughly 60 g, 0.6 moles) was added over a period of 30-60 minutes to achieve a pH of >7.5. The temperature of the mixture was then increased to 53-57° C., and maintained at that temperature for 30 minutes. Water (237.4 mL, 13.18 moles) was then added over a 25-minute period. Afterward, the mixture was ramp-cooled to 19-22° C. over a 2-hour period, and then maintained at that temperature for an additional 30 minutes. The slurry was filtered, and the cake was washed with water (237.4 mL, 13.18 moles) and then with acetonitrile (237.4 mL, 4.53 moles). The resulting white solid was dried in a vacuum oven at 50° C. to afford 76.0 g of product. A sample was collected for analysis.

### Step E. Synthesis of 2-amino-6-ethyl-6,7-dihydro-5H-indeno[5,6-d]thiazole

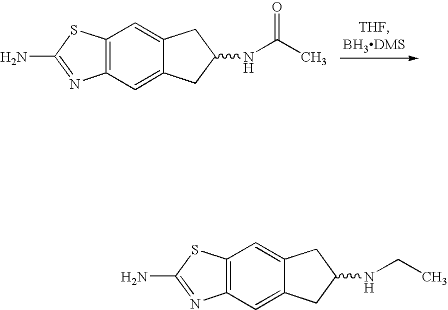

2-Amino-6-acetylamino-6,7-dihydro-5H-indeno[5,6-d]thiazole (16.3 g, 60.0 mmoles) and tetrahydrofuran (296.7 mL, 3.65 moles) were charged to a jacketted reactor fitted with an overhead stirrer, condenser, temperature probe, and N₂ inlet. The mixture was stirred while being heated to a temperature of >55° C. Borane-methyl sulfide complex (25.13 mL, 269.89 mmoles) was added over a period of >1 hour while maintaining the temperature at 55-60° C. A sample was collected for analysis.

The mixture was cooled to <45° C. Afterward, water (74.17 mL, 4.12 moles) was added over a period of 90-120 minutes while stirring the mixture and maintaining a temperature of 40-45° C. Agitation was slowed after ⅕ of the water had been added due to the formation of a large lump of solid after ¼ of the water addition. The lump eventually broke down to form a colorless solution. Following the addition of the water, HCl (17.97 g, 179.9 mmoles) was charged to the reactor over a 30-minute period while maintaining the temperature at 40-45° C. Afterward, the mixture was heated to a temperature of >55° C., and then maintained at that temperature for 30 minutes. At this point, the mixture was a hazy, biphasic colorless solution. A sample was collected for analysis. Agitation was then stopped, and the two phases were allowed to separate over a period of >5 minutes. The lower aqueous phase was a yellow solution, and the upper THF phase was a colorless solution. The THF phase was discarded. Stirring was then initiated to the aqueous phase. Water (37.1 mL, 2.06 moles) and acetonitrile (37.1 mL, 707.5 mmoles) were then added over a 50-minute period while maintaining the mixture at a temperature of 50-60° C. Potassium hydroxide (22.43 g, 179.9 mmoles) was then added over a 2-hour period while stirring the mixture and maintaining a temperature of 50-60° C. At a pH of 3.5-4, a pale yellow solid precipitated. After all the potassium hydroxide was added, the pH was 12 and the mixture was a fine yellow suspension. The suspension was cooled to 20° C. over a 2-hour period, and then filtered. The resulting pale yellow cake was washed with water (14.83 mL, 823.4 mmoles) and acetonitrile (14.8 mL, 283.0 mmole), and then washed again with water (26.7 mL, 1.48 moles) and acetonitrile (2.97 mL, 56.6 mmoles). The resulting white solid was dried under vacuum at 50° C. to afford 52.9 g of product. A sample was collected for analysis.

### Step F. Isolation of (R)-N*6*-ethyl-6,7-dihydro-5H-indeno[5,6-d]thiazole-2,6-diamine from racemate

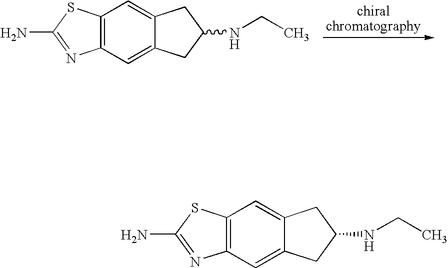

2-Amino-6-ethyl-6,7-dihydro-5H-indeno[5,6-d]thiazole (6 g) was dissolved in a methanol/diethylamine (240 mL, 100:0.1) solvent. The resulting solution was filtered and injected into an HPLC column (Chiralpak Iowa, Daicel Chemical/Chiral Technologies) under the following conditions:

| | |
|---|---|
| Internal column diameter | 200 mm |
| Column length | 200 mm |
| Packing | Chiralpak IA, 20 micron |
| Mobile Phase solvent mixture | acetonitrile/diethylamine (100:0.1 v/v) |
| Mobile phase flow rate | 72 L/hr |
| Run time | Approximately 23 minutes (injections |
| | were stacked such that a further injection |
| | initiated before elusion of the previous |
| | injection was complete) |

The retention time for the (R)-N-*6*-ethyl-6,7-dihydro-5H-indeno[5,6-d]thiazole-2,6-diamine product was approximately 20 minutes. This produced a product having a purity of ≧97.6% ee. A 5.6 L fraction containing this purity was evaporated to dryness on a rotary evaporator. The resulting solid residue was re-dissolved in isopropanol (3.57 L) and used directly in the following purification.

### Step G. Purification of (R)-N*6*-ethyl-6,7-dihydro-5H-indeno[5,6-d]thiazole-2,6-diamine

The crude (R)-N*6*-ethyl-6,7-dihydro-5H-indeno[5,6-d]thiazole-2,6-diamine solution from Step F (3.42 kg, 3.42 L, 0.06 M, 205.2 mmoles) was charged via a 20 μm screen to a 3 L jacketed vessel having a condenser, mechanical agitation, a temperature probe, and N₂ inlet. The lines were then washed with isopropyl alcohol (95.8 mL, 1253 mmoles), which, in turn, also was added to the vessel. After initiating agitation and preparing the vessel for reduced-pressure distillation, the pressure was reduced to 600 mbar and the temperature was increased to 75-80° C. to begin distillation. The distillation was stopped when the solvent volume was reduced to 13 rel vols (650 ml). Afterward, the vessel was prepared for reflux return of solvent, and the mixture was cooled to a temperature of 70-72° C. Pure (R)-N*6*-ethyl-6,7-dihydro-5H-indeno[5,6-d]thiazole-2,6-diamine (383.0 mg, 1.64 mmoles) was seeded in 2 portions, with the second seeding occurring after the first seeding equilibrated. The resulting slurry was held at a temperature of 70-72° C. for an additional 2 hours, cooled to 20° C. over a 4-hour period, and then held at that temperature for 10 hours. A sample was collected for analysis. (In some instances (particularly on scale up), the slurry was held at 20° C. for an additional 6 hours, and, in some such instances, also was subsequently heated to 50° C. for 3 hours. Such additional step(s) tended to produce a desired crystalline structure, and may be repeated. Their use depended on, for example, variations in equipment, cooling rates, scale of process, etc.) The slurry was then cooled to 10° C. over a 1-hour period, and then held at that temperature for at least 2 hours. Afterward, the slurry was filtered under low vacuum to deliquor the cake. Isopropyl alcohol (37.64 g, 626.3 mmoles) was first used to wash out the remaining solids from the vessel at a temperature of 10-13° C., and then passed through the same filter. The resulting combined cake was then dried to a constant weight in a vacuum oven at 50° C.

The words “comprise,” “comprises,” and “comprising” in this patent (including the claims) are to be interpreted inclusively rather than exclusively. This interpretation is intended to be the same as the interpretation that these words are given under United States patent law.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

We claim:

1. A compound or a pharmaceutically acceptable salt thereof, wherein the compound corresponds in structure to formula I:

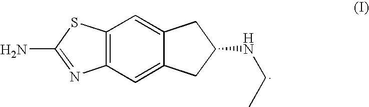

2. A composition, wherein the composition comprises a compound or pharmaceutically acceptable salt of claim 1.

3. A composition of claim 2, wherein the composition comprises a therapeutically effective amount of a compound of formula I or a pharmaceutically acceptable salt thereof.

4. A compound or salt of claim 1 for use as a medicament.

5. The use of a compound or salt of claim 1 in the manufacture of a medicament for treating a dopamine-related central nervous system disorder.

6. The use set forth in claim 5, wherein the dopamine related central nervous system disorder is selected from the group consisting of schizophrenia, Parkinson's disease, Tourette's Syndrome, hyperprolactinemia, drug abuse, major depressive disorder, and bipolar disorder.

7. A method of treating a dopamine related central nervous system disorder in a patient in need of such treatment, wherein the method comprises administering a therapeutically effective amount of a compound or salt of claim 1 to the patient.

8. The method of claim 7, wherein the dopamine related central nervous system disorder is selected from the group consisting of schizophrenia, Parkinson's disease, Tourette's Syndrome, hyperprolactinemia, drug abuse, major depressive disorder, and bipolar disorder.

9. A method for preparing the compound or salt of claim 1, wherein the method comprises reacting N-((R)-6-ethylamino-6,7-dihydro-5H-indeno[5,6-d]thiazol-2-yl)-benzamide.2HBr with hydrobromic acid under conditions sufficient to yield (R)-N*6*-ethyl-6,7-dihydro-5H-indeno[5,6-d]thiazole-2,6-diamine.2HBr.

10. The method of claim 9, wherein the method further comprises:
dissolving resultant (R)-N*6*-ethyl-6,7-dihydro-5H-indeno[5,6-d]thiazole-2,6-diamine.2HBr in water, and
adding base to precipitate out (R)-N*6*-ethyl-6,7-dihydro-5H-indeno[5,6-d]thiazole-2,6-diamine.

11. The method of claim 9, wherein the method further comprises preparing N-((R)-6-ethylamino-6,7-dihydro-5H-indeno[5,6-d]thiazol-2-yl)-benzamide.2HBr by a method comprising reacting hydrobromic acid and triisopropylsilane with ((R)-2-benzoylamino-6,7-dihydro-5H-indeno[5,6-d]thiazol-6-yl)-ethyl-carbamic acid benzyl ester.HBr under conditions sufficient to yield N-((R)-6-ethylamino-6,7-dihydro-5H-indeno[5,6-d]thiazol-2-yl)-benzamide.2HBr.

12. The method of claim 11, wherein the method further comprises preparing ((R)-2-benzoylamino-6,7-dihydro-5H-indeno[5,6-d]thiazol-6-yl)-ethyl-carbamic acid benzyl ester.HBr by a method comprising reacting a solution of ((S)-5-amino-indan-2-yl)-ethyl-carbamic acid benzyl ester with a solution of benzoyl isothiocyanate under conditions sufficient to yield ((R)-2-benzoylamino-6,7-dihydro-5H-indeno[5,6-d]thiazol-6-yl)-ethyl-carbamic acid benzyl ester.HBr.

13. The method of claim 12, wherein the method further comprises preparing ((S)-5-amino-indan-2-yl)-ethyl-carbamic acid benzyl ester by a method comprising reacting a solution of [(S)-5-(benzhydrylidene-amino)-indan-2-yl]-ethyl-carbamic acid benzylester with hydroxylamine hydrochloride and NaOAc under conditions sufficient to afford ((S)-5-amino-indan-2-yl)-ethyl-carbamic acid benzyl ester.

14. The method of claim 13, wherein the method further comprises preparing [(S)-5-(benzhydrylidene-amino)-indan-2-yl]-ethyl-carbamic acid benzyl ester by a method comprising:
reacting tris(dibenzylideneacetone)dipalladium(0), 2-dicyclohexylphosphino-2′,4′,6′-triisopropylbiphenyl and toluene to yield an intermediate solution; and
adding sodium tert-butoxide, an N₂-purged solution of ((S)-5-bromo-indan-2-yl)-ethyl-carbamic acid benzyl ester, and benzophenone imine under conditions sufficient to yield [(S)-5-(benzhydrylidene-amino)-indan-2-yl]-ethyl-carbamic acid benzyl ester.

15. The method of claim 14, wherein the method further comprises ((S)-5-bromo-indan-2-yl)-ethyl-carbamic acid benzyl ester by a method comprising:
reacting a cooled solution of ((S)-5-bromo-indan-2-yl)-carbamic acid benzyl ester with NaH with stirring;
adding ethyl iodide with stirring; and
extracting ((S)-5-bromo-indan-2-yl)-ethyl-carbamic acid benzyl ester.

16. The method of claim 15, wherein the method further comprises preparing ((S)-5-bromo-indan-2-yl)-carbamic acid benzyl ester by a method comprising reacting (S)-5-bromo-indan-2-ylamine (1R)-(−)-10-camphorsulfonate salt with benzyl chloroformate.
